(19) United States
(12) Patent Application Publication
(10) Pub. No.: US 2010/0239668 A1
(43) Pub. Date: Sep. 23, 2010

(54) SEGMENTED PHARMACEUTICAL DOSAGE FORMS

(76) Inventors: Allan S. Kaplan, Boca Raton, FL (US); Lawrence Solomon, Miaim Beach, FL (US)

Correspondence Address: ACCU-BREAK TECHNOLOGIES, INC., 1000 SOUTH PINE ISLAND ROAD, SUITE 230, PLANTATION, FL, 33324, US

(21) Appl. No.: 12/305,747

(22) PCT Filed: Jun. 19, 2007
(86) PCT No.: PCT/US07/71567

§ 371 Date: Dec. 19, 2008

Related U.S. Application Data
(60) Provisional application No. 60/815,556, filed on Jun. 19, 2006.
(60) Provisional application No. 60/815,557, filed on Jun. 20, 2006.
(60) Provisional application No. 60/818,810, filed on Jul. 6, 2006.

(51) Int. Cl. A61K 9/44; A61K 9/22; A61K 31/551; A61K 31/60; A61K 31/5415; A61K 31/415; A61K 31/192; A61K 31/405; A61K 31/708; A61K 33/00; A61K 31/135; A61K 31/455; A61K 31/4166; A61K 33/14; A61K 31/522; A61P 43/00

(52) U.S. Cl. 424/467; 424/464; 424/468; 514/220; 514/165; 514/226.5; 514/406; 514/570; 514/420; 514/46; 424/715; 514/646; 514/356; 514/391; 424/679; 514/263.34

(57) ABSTRACT

Described is a layered or segmented controlled release pharmaceutical tablet adapted for separating one segment from another by breaking through a segment.

### FIELD OF THE INVENTION

The invention involves layered pharmaceutical tablets comprising a layer or segment containing a drug and a second layer or segment comprising a composition that is (a) lacking or substantially free of a drug, (b) containing the same drug as in the first segment but as part of a different formulation, such as in a different strength or concentration or with different excipients, or (c) containing at least one different drug. Methods concerning the manufacture and use of the subject dosage forms are also included as part of the invention.

More specifically, the subject invention concerns novel dosage forms and methods for providing divisible, controlled release pharmaceutical products which in preferable embodiments can provide accurate and consistent divided doses. The dosage form can comprise a segment which has a score, which can be a deep score.

### BACKGROUND OF THE. INVENTION

The invention derives from the need to solve common problems within the pharmaceutical industry, such as inaccurate or inconsistent dose division that can occur upon breaking of a dosage form, or the lack of accurate dosing flexibility for one or more drugs contained in a combination dosage form. For controlled-release dosage forms, these problems can be exacerbated by the fact that the controlled-release characteristics can be detrimentally modified or lost altogether if the whole dosage form is broken or divided to provide a lower or separate dose from the whole.

It is known that pharmaceutical tablets are commonly broken to modify the dose provided by a whole tablet. Breaking of tablets into smaller portions by patients has been determined to be an imprecise method of adjusting dosage. Tablets are often produced with a score to ostensibly aid breaking, but it has been well-documented that standard scoring of tablets does not provide precision in dosage adjustment. Many drugs, such as warfarin, require dosage adjustments during treatment.

A second problem arising from dividing or breaking a dosage form relates to combination drug products, i.e., single or unitary dosage forms containing two or more active ingredients. Combination dosage forms are typically produced as homogeneous mixtures or as capsules. Therefore, a physician prescribing such combination products may not adjust the dose of only one of the active ingredients in the combination product without a consequent proportional adjustment to the dose of the other active ingredient(s) contained within the combination drug product. Conventional dosage forms containing drug combinations can thus be disadvantageous due to the inflexibility of dealing with changing circumstances, such as fluctuating blood pressure or the appearance of side effects to a drug.

Even if the active ingredients in a combination product are layered separately within a fixed-dose tablet, breaking of such dosage forms—especially if scored in a conventional pattern on a top face of a standard wider-than-tall tablet—results in breaking through all layers, thus dividing all active ingredients relatively proportionally. However, breaking of conventional layered tablets may not divide the doses accurately or precisely because of chipping or crumbling that can occur during breaking, or because of uneven breaking of the dosage form resulting in unequally subdivided doses.

Moreover, the known layered combination dosage forms are provided primarily to address the problem of a physical or chemical incompatibility between the different layers containing active drug. In such case, it is specifically taught that the “separating layer” be as limited in size (thickness) as is necessary to separate the incompatible layers.

Certain layered dosage forms have been described in the patent literature. For example, U.S. Pat. No. 5,738,874 to Conte, et al. describes a multi-layer controlled release tablet having a first layer comprising an immediate release drug composition, a second layer comprising a slow release drug composition, and a third layer comprising a barrier composition to modify release of drug from the layer adjacent thereto. This third, drug-free layer, even if interposed between the drug-containing layers, is not useful for facilitating breakage or splitting of the tablet, nor does it provide the advantage of being able to separate one active layer from another prior to administration in order to adjust or titrate a dose of only one of the drugs in a fixed-dose combination dosage form.

Published U.S. Patent Application 2005/0019407A1 to Sowden, et al. describes a composite dosage form which has first and second portions joined at an interface. These dosage forms have a first molded material and a second compressed material. There is no disclosure of any modification of the described dosage forms that would facilitate the breaking of the dosage forms into a subdivided form for the advantageous purpose of dose adjustment or titration.

U.S. Pat. No. 6,602,521 describes a multiplex drug delivery system containing at least two immediate release drug dosage packages enveloped by a scored, extended release compartment. There is no teaching from the disclosure of this patent of a controlled release compartment which does not envelop the immediate release compartments.

Concerta® is believed to be the only commercially available pharmaceutical product that is produced as a taller-than-wide dosage form. Concerta® is a layered tablet, having a semi-permeable membrane and utilizes the OROS® system for its controlled release characteristics. The manufacturer's directions for the use of Concerta® specify that the tablets should never be broken. Concerta® does not include an inactive layer interposed between two active controlled-release layers.

No controlled release dosage form is provided in the prior art that fully addresses the problems facing the pharmaceutical tablet industry. The present invention, as disclosed herein, can overcome or alleviate these problems and can provide additional advantages and address additional problems by making available a segmented (e.g., layered) compressed tablet having a specific segment useful as a breaking region.

### SUMMARY OF THE INVENTION

The subject invention relates, in its preferred embodiment, to a controlled-release pharmaceutical tablet advantageously adapted for being capable of separating, prior to administration, one active segment from another without affecting the release rate of drug from any resultant tablet portion. The present invention in this preferred embodiment concerns at least two pharmaceutically active controlled-release (CR) formulations, e.g., CR granulations, compressed beads or pellets, or matrix compositions comprising at least one active drug configured as different parts or segments of a compressed tablet. The active ingredients can be the same or different. The tablet further comprises at least one inactive layer or segment in contact with each of the active segments, and serving as a breaking region for the tablet. This configuration allows for accurate and precise separation of the active segments (and the active drugs contained therein) without affecting the release rate of drug from the active segment. In a preferred embodiment having three or more layers, the inactive segment is a separating layer or segment interposed between two active CR segments and is dimensioned (e.g., has a height, as defined or referred to herein) so as to allow breakage transversely through the separating layer without consequent breakage of other tablet segments.

In another preferred embodiment for a tablet having two layers, the invention involves an outer (upper or lower) segment that is contiguous with a controlled-release segment containing an active drug, such as a matrix composition, in which said matrix composition is preferably scored or otherwise is provided with a separation mark such as a score. Dividing or scoring the active layer(s) completely therethrough can provide a substantially hi-layered tablet having three or more segments.

Where the active ingredients are different, thus providing a combination drug product, a preferred embodiment is to provide each active ingredient in a separate segment. Preferably, a first active ingredient of the combination product is formulated in a composition used to form a first segment, and the second active ingredient is formulated in a composition used to form a second segment. The composition containing each active ingredient can be a controlled release composition or an immediate release composition.

The preferred dosage forms of the subject invention can include a segment which separates, or is interposed between, two active segments, all contained within a single dosage form. The interposed, or “middle,” segment can be substantially drug-free (referred to herein as an “inactive segment”) or can contain one of the first or second drugs in a different concentration, or can contain a third active drug. This separation of active segments can be provided in order to separate two incompatible drugs or drug-containing compositions, or can provide a breaking region between two segments containing the same or different active ingredient(s) which can allow for accurate division of the dose.

A benefit of such matrix controlled release compositions configured in a tablet according to the subject invention is the advantage of the tablet being provided with a segment that may be broken without affecting any active segment, e.g., a matrix formulation, and thus the release of the active drug therefrom.

One object of the invention is to provide a tablet which k breakable into smaller portions and consequent smaller doses wherein, when broken, each portion retains a constant exposed surface area for the portions comprising active ingredient. A conventional matrix tablet, having active drug homogenously distributed throughout the tablet will exhibit release rates for that active drug that differ if the tablet is broken because the surface area of the tablet changes after breaking. A tablet of the subject invention advantageously allows for maintaining constant dissolution or pharmacokinetic properties for drug release whether the tablet is administered whole or as a broken portion because exposed surface area of the controlled release portion of the tablet remains constant.

The subject invention provides a pharmaceutical tablet with at least two different vertically disposed layers which, when compressed, form tablet segments in which at least one segment comprises a drug or drugs in an “altered release”, or “controlled release” formulation. Where the tablet includes three or more segments, at least two segments, in a preferred embodiment, contain the same concentration or amount of a drug or drugs in a substantially similar formulation and the third segment comprises a formulation that lacks a pharmacologically effective amount of a drug.

Such dosage forms of the subject invention comprising at least one segment which is formulated as a controlled release composition are therefore considered as controlled release dosage forms. Thus, for example, controlled release tablets of a three-segment preferred embodiment of the subject invention comprise longitudinally compressed tablets having two “outer” layers or segments and an “inner” layer disposed therebetween. The outer segments preferably comprise the “upper” or “top” segment, and a “lower” or “bottom” segment. These layered or segmented configurations for the tablets of the subject invention result in tablets that, as a whole in their final dosage form, are non-homogeneous.

The interposed inner layer preferably contacts another layer or segment at only one interface, and does not encompass or envelop any other layer or segment of the tablet. For a controlled release dosage form comprising this preferred three segmented configuration as described, an inner inactive segment that is impervious to egress from said one or more controlled release composition by a drug is considered part of the invention.

Another preferred embodiment of the invention is a pharmaceutical tablet having two or more segments, has a top and a bottom, and has a height that exceeds the width of said tablet, i.e., the tablet is taller than it is wide. The “height” of the tablet refers to its measurement vertically, from the top to the bottom of said tablet as it is positioned in the tablet die when fully compressed. The “width” of the tablet refers to its measurement at its greatest horizontal dimension. The terms “vertical” and “horizontal” (“horizontal” is also referred to as “transverse”) axes of the tablets of the invention are determined by the orientation of the tablets in the tablet die when fully compressed, as well as the order of entry of granulations into the die.

Taller-than-wide tablets of the invention are shaped to be more easily broken through a tablet's theoretical vertical axis (i.e., in a horizontal direction) than are conventional, currently manufactured tablets having a “wider than tall” configuration. A preferred use of tablets of the invention is to break through an inactive segment of the tablet, such as an interposed inner segment of a three-segment tablet of the subject invention, without breaking through a segment above or below said interposed segment.

Tablets of the invention are adapted to be useful not only as whole tablets but also to be breakable into subunits known herein as “tablettes”, with accurate dosing both as whole tablets and in tablette form. “Tablette” is the term used herein to refer to the resultant portions of a tablet of the subject invention following breaking of those tablets to provide lower dosages. The breaking step can apply to the whole tablet, e.g., a whole dosage form broken once to form two “tablettes,” or can apply to further breaking of a tablette itself, e.g., breaking a half-dose “tablette” (from a whole tablet) into a quarter-dose “tablette.” The invention achieves these ends by utilizing in many of its preferred embodiments a segment that comprises a granulation or other composition that can be substantially free of active drug (an “inactive granulation” that comprises an “inactive segment”).

It is one primary object of the invention to create controlled release pharmaceutical tablets adapted to be broken when it is desired to create a lower dosage of a drug or drugs present in the whole tablet, by allowing breaking through a segment that prevents or significantly retards release of drug contained within a segment of the tablet comprising a controlled release composition.

It is another primary object of the invention to apply the invention both to accurate dosing of single agent products and to combination products.

With regard to the use of the subject invention for combination products, it is understood that a mixture of drugs within one granulation acts as a single drug from the standpoint of the separability of one segment from another. In a preferred embodiment in which, for example, Drug A is present in a therapeutically effective quantity in an upper segment, an inner segment that lacks a pharmacologically effective quantity of any drug is interposed between two outer (i.e., top and bottom or upper and lower) segments, and Drug B is present in a therapeutically effective quantity in a lower segment, then the invention can be useful in the situation that the height and especially the “effective height” of said inner segment is great enough to allow said inner segment to serve as the breaking region of said tablet substantially without breaking through either outer segment. The prior art, however, is such that novelty for the subject invention requires no minimum height of said inner segment if, in said tablet, all ingredients of the upper and lower segments are physically and chemically compatible with each other.

In the specialized situation in which there is an incompatibility between components of said outer segments, then the prior art is such that any inner “separating” segment should be limited in height to the minimum needed to eliminate the presence of any of said incompatibilities, for such reasons as to minimize the size of the tablet as a whole or to minimize cost. In that case, the invention remains novel in any of its more preferred forms, in which there is provided, relative to a quantity provided solely to separate incompatible layers, as is known, an excess quantity of said inner separating segment to allow it to be broken.

### BRIEF DESCRIPTION OF THE DRAWINGS

FIG. 1 shows a cross-section of a three-segment tablet of the subject invention.

FIG. 2 shows a cross-section of a scored two-segment tablet of the subject invention.

FIG. 3 shows a cross section of a two-segment tablet of the subject invention having a score which is formed completely through the active segment and into the inactive segment.

FIG. 4 shows a different embodiment of a three-segment tablet of the subject invention.

FIG. 5 shows a three-segment tablet of the subject invention having a score in the middle segment.

FIG. 6 shows a variation of the three-segment tablet of FIG. 4 wherein the tablet comprises three active segments plus two inactive segments where each inactive segment is a harrier interposed between active segments.

FIG. 7 shows a five-segment tablet as in FIG. 6, having a score in the middle active segment.

FIG. 8 shows a further embodiment of a five-segmented tablet of the invention comprising two different active compositions forming the bottom two segments, two different active compositions forming the top two segments, and an inactive barrier segment interposed between the to two and bottom two segments.

FIG. 9 shows a five-segment tablet as in FIG. 8, having a score in the middle barrier segment.

FIG. 10 shows a seven-segment variation of the five-segment tablet of FIG. 8 where the two top active segments are separated by an additional interposed inactive segment and the two bottom active segments are separated by an additional interposed inactive segment.

FIG. 11 shows a cross section of a hi-layer tablet of the subject invention having a score which is formed completely through one active segment and into a second active segment which serves as a base layer or segment of the tablet.

FIG. 12 shows a variation of the hi-layer tablet of FIG. 11 wherein a third inactive or barrier segment is interposed between the first and second active segments.

All of the whole tablets referenced above comprise at least two compositionally different segments wherein at least one segment can comprise an intrinsically altered- or controlled-release characteristic.

### DETAILED DESCRIPTION OF THE INVENTION

The subject invention concerns, in its preferred embodiments, a novel tablet configured to provide substantially accurate or precise divided doses of at least one active ingredient when broken into tablet portions. In one preferred embodiment, a tablet of the subject invention is layered and comprises three or more segments wherein at least one of the segments comprises an active pharmaceutical ingredient (API) formulated as an altered or controlled release composition. More preferably, a tablet of the subject invention is configured to provide a top segment comprising an API formulated as a controlled release composition, a bottom segment comprising an API formulated as a controlled release composition, and a middle segment, interposed between the top and bottom segments. The composition of the middle segment preferably comprises a different API than is present in the top and bottom segments, or contains substantially no API (which includes API in amounts that may be detectable but are substantially pharmaceutically inactive).

It is understood that the top and bottom segments can comprise the same API in substantially the same concentrations or amounts, or can comprise different APIs or different APIs in the same or different concentrations or amounts. The whole tablets described and shown herein preferably comprise at least one segment that intrinsically has an altered-release characteristic. Intrinsic altered release characteristic refers to the release property of the composition itself i.e., altered or controlled release (as compared to immediate release) of drug or drugs from the composition without the use of a device or composition that is extrinsic to the segment composition for altering or controlling said release, such as a coating or membrane or the like. The preferred tablets of the subject invention are therefore of altered release or controlled release nature due to the composition of the one or more segments that is intentionally formulated to have such an altered release property. It should be understood, however, that one or more, or even all of the segments of the subject invention can comprise immediate release compositions, and such dosage forms are considered part of the invention.

The terms “altered release” and “controlled release” are contemplated to include a dosage form or composition that has the property of releasing active ingredient from the dosage form at a modified or “altered” rate relative to the rate of release of drug from a conventional “immediate release” formulation, as would be well understood in the art. Therefore, the term “altered release” includes “controlled release”, “delayed release”, “extended release”, “long-acting”, “modified release”, “slow release”, “sustained release”, “time release”, and the like, all of which are understood to refer to a release which is later or slower than “immediate release.”

A release which is delayed due formulation with enteric or other materials, though releasing immediately following the delay, is understood in the art to be a type of “controlled release”, and is so considered for purposes of the subject invention. “Slow release”, “extended release”, “long-acting”, “sustained release”, “time release”, and the like, are generally recognized as being synonymous and may be used interchangeably herein to designate an “altered release” or “controlled release” formulation which is not “delayed release”.

“Altered release” may also mean a release rate which is more rapid than a conventional immediate release tablet, for example, a rapid-dissolve tablet or quick-dissolve tablet, which dissolves in the mouth or buccal area before being swallowed, as is also well known and understood in the pharmaceutical industry.

The term “intrinsically altered release” refers to a controlled release property of a pharmaceutical composition, e.g., a granulation or matrix composition, whereby the release rate of drug or drugs from that composition is affected by the ingredients or excipients of that composition and not a device or composition that is extrinsic to that composition, e.g., a coating or membrane disposed onto or formed around the composition.

The terms “active agent,” “drug,” “active drug,” “active pharmaceutical agent,” “active pharmaceutical ingredient,” “API” and “pharmacologically active agent” may be used interchangeably herein to refer to a chemical material or compound which, when administered to an organism (human or animal) induces a pharmacologic effect, and which includes prescription and non-prescription pharmaceutical compounds, and such substances as pharmacologically effective doses of vitamins or co-factors, minerals, including biologically effective salts, and the like.

By convention herein, the term “segments” may be used in place of “layers” in general in discussing the finished tablets of the invention, for reasons that are explained below. In addition, for convenience of reference and consistency throughout this specification, the descriptions herein may refer to the segments as comprising or utilizing a particular “granulation”. Such term is not limited to the formation of granules, per se, as in a wet granulation process. Other formulation compositions, for example, homogeneous mixtures or blends used in direct compression matrix formulations, coated or uncoated beads or pellets used in compressed tablets, or like compositions as are well known in the art and suitable for use in conventional layered, compressed tablet technologies, can be readily substituted for such “granulations” and are considered within the scope of the invention. It is expressly intended that the subject invention include each of these alternatively available and well known compressible formulation technologies.

A segment represents the entirety of a substantially homogeneous contiguous part of a tablet. A segment may be formed from more than one layer, however: if two substantially identical granulations entered the tablet die successively, with the second entering directly after and onto the first, such as at two successive filling stations during automated high-speed tablet manufacture, then the two granulations would each form a separate layer after entering, but when compressed, they would comprise one segment. A segment therefore is a basic unit of how the tablets of the invention prove useful. If, however, two different active drugs, or different salts of the same active drug, were compressed one on top of the other, they would form two segments. Granulations comprising the same active drug but with dissimilar excipients would also form two segments if one granulation were compressed onto another.

A segment formed by a plurality of layers that are formed from substantially identical granulations is called a compound segment. Compound segments may prove useful in situations of relatively large quantities of an inactive granulation, or granulation containing a drug or drugs, so that two or more consecutive fills (“feeds”) of substantially identical granulation may occur.

A layer formed from a granulation that is neither disposed upon nor under (i.e., does not adjoin and is not contiguous with) a substantially identical granulation is a simple segment. A non-compound segment is a simple segment.

As used herein, such terms as “horizontal” (“transverse”) and “vertical” when used in relation to a tablet, are based on the spatial orientation of the tablet as, and after, it is produced in a die, but before removal or ejection from the die. Current methods of manufacture produce tablets with one granulation entering the die on top of another, so that tablets of the invention produced in such a manner comprise one or more top (outer) segments, one or more bottom (outer) segments, and optionally one or more middle (inner) segments. A segment that is not a top or bottom (i.e., outer) segment is considered to be an inner segment.

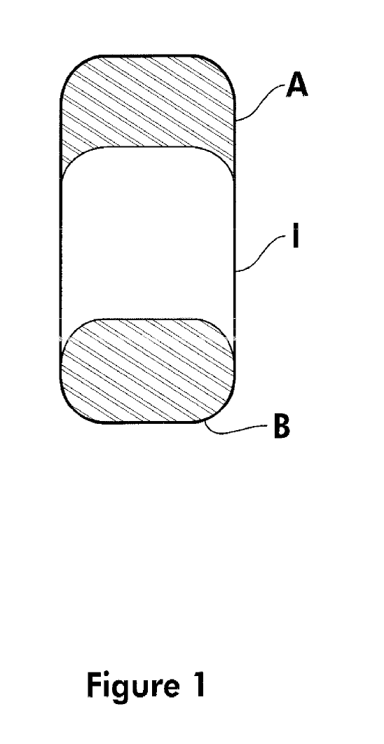

If separate granulations were to be sequentially placed in a die horizontally (side-to-side) and not vertically as is currently the practice, then the tablets so produced would be within the scope of the present invention because the same resultant product would be produced by the horizontal compression process. When the tablet of FIG. 1, for example, is laid on a flat table, it will tend to lie lengthwise at right angles to the manner in which it is formed in the die, so that if the three segments were all different colors, then the segments would appear to be arranged not vertically (one on top of the other), but rather horizontally (side-to-side). For consistency of terminology, such segments nonetheless are considered herein to be disposed vertically on top of each other.

In any configuration of a tablet according to the subject invention, the lateral parts of any outer or inner segment have an externally exposed surface.

The term “undetectable amount” means that when using conventional analytical techniques such as high performance liquid chromatography (HPLC), nuclear magnetic resonance imaging (NMRI), and the like, the presence of an active compound can not be identified. The term “pharmacologically ineffective amount” means an amount of a drug or drugs that has no measurable pharmacological effect. Due to the conditions under which high speed automated tabletting equipment are operated, mixing of different granulations may occur during tablet formation which may cause material such as drug substance present in one granulation to appear in a layer or segment where it was not intended to be placed.

The term “relatively inactive segment” refers to a segment that either contains an undetectable amount of any drug or contains a decreased concentration of any drug or drugs contained in another segment or segments in a pharmacologically effective quantity. The term “decreased concentration” means that the concentration of a drug or drugs in said relatively inactive segment is no more than 80% that of said drug or drugs in another segment, more preferably no more than 20% of said other segment's drug or drugs concentration; most preferably said ratio is no more than 5%, however. The concentration of a drug or drugs in a segment means, herein, the ratio, on a weight to weight basis, of the drug or drugs in said segment to the total weight of said segment, which includes said drug or drugs and inactive excipients.

Segments containing pharmaceutically inactive ingredients, active compositions identical to another segment, or active compositions different from another segment, as well as combinations of active compositions, can also be included as part of the tablet to provide four or more, preferably about four to nine, layered segments within the tablet. In these further embodiments, certain inner segments can comprise active compositions, which are interposed between an outer and inner segment, or between two inner segments. The number of layers or segments is limited only by the layer press equipment available, and the desirability of the finished product.

Tablets of the invention are preferably produced on a layer press, such as a tri-layer or five-layer high-speed press, such as the Korsch 900 manufactured by Korsch AG of Germany, Remington's Pharmaceutical Sciences 20th Ed., Mack Publishing Co., Easton, Pa. (2000), Chapter 45, which is incorporated by reference, describes the various techniques utilized in making compressed tablets. In summary, tablets of the subject invention are formed by compressing, e.g., vertically, at least two different pharmaceutical formulation compositions, e.g., granulations or matrix compositions, configured as separate layers or tablet segments. Preferably, the subject tablets comprise three vertically disposed segments. Embodiments of the subject invention include, but are not limited to, a vertically compressed tablet having a height greater than its width (a “taller than wide” tablet), and a unitary segmented tablet.

As an example of a method of manufacture of a preferred tablet of the invention, first, a granulation containing a pharmacologically effective dose of a drug enters the die and is tamped to form a first segment. Second, a granulation lacking a drug (an “inactive granulation”) or a granulation comprising a drug different than in the first granulation enters the die and is tamped. This second granulation creates a part of the tablet that can be identified and broken through so that the segments contiguous therewith are not broken through. Last, a third granulation containing a pharmacologically effective quantity of a drug enters the die, is optionally tamped, and then a final compression is performed to form a third segment and a final compressed tablet occurs. While the above description refers to a three-segment tablet, it would be understood that additional layers or segments may be added at any step in order to provide a desired layered or segmented tablet. For example, additional active or inactive layers can be interposed between active segments as described, forming a tablet comprising from four to nine layers or segments, depending on the capacity of the layer tablet press being used. One or all segments may individually have a width greater than height, the tablet as a whole has a height that exceeds its width.

A layer is produced by introducing an amount of an individual granulation into a tablet die to fill at least a part of the die. A layer is considered to be present whether it is the form of an un-tamped, tamped or fully compressed granulation. Suitable dimensions for tablets (intended for adult human use) according to the invention are without limitation height: 6 to 24 mm.

The tablet embodiments of the subject invention can comprise a separation mark or score. For tablets manufactured as vertically disposed layers forming the tablet segments, a score may be optionally placed in the side of said tablet subsequent to tablet formation, preferably transversely. Alternatively, after tablet formation, a printed line or other forms of indicia such as dotted lines, symbols or perforations may be placed on or in the surface of the tablet, all of which serve the purpose of allowing identification of said tablet's desired breaking region from the standpoint of effecting accurate separation of the parts of a tablet containing isolated doses of drug. Other means of aiding identification of a region of potentially desired tablet breaking may be utilized such as the use of contrasting colors in different segments.

Additionally, the compressed tablet can be further processed to provide an inert covering, e.g., a gelatin capsule or a sachet. In use, the covering can be cut away or otherwise removed, such as by twisting apart a conventional gelatin capsule, removing the tablet therein and dividing the tablet as described herein for a non-encapsulated embodiment. Alternatively, a separation mark provided on the capsule or sachet can guide a user to divide the tablet or its covering at a designated site in order to effect an accurate splitting of a tablet of the subject invention. The covering can advantageously be useful to minimize or prevent confusion on the part of the patient user viewing a segmented or layered tablet of the subject invention. The tablet may be coated in a variety of ways, without limiting the invention.

In certain of the preferred tablets of the invention, a layer (and the granulation from which it is derived) will not need to be placed on top of or below (e.g., adjoining, or contiguous with) a substantially identical layer (or granulation). In such a case, one layer will give rise to the sub-type of segment that is referred to as a “simple” segment. The use of the term “segment” allows a segment to be simple or compound.

Because the tablets of the invention have been adapted to be broken if and when desired, a term for the major fragments resulting from said breaking has been coined. The inventors use the term “tablette” in this regard. An example of tablette formation is as follows: a standard single-scored, mono-layer, homogeneous pharmaceutical tablet is broken. Said breaking produces two major fragments, each of which is called a tablette. Some chipping and crumbling, which are preferably minor in amount, may occur. In the segmented, layered tablets of the invention, to utilize the invention properly may make it advantageous to place a score transversely into a segment, such as an inner segment, as may be done with an instrument such as a file. Successfully breaking said tablet through said score will result in two tablettes, representing the two major fragments of the tablet and not including smaller fragments such as crumbs or chips.

Of the many tablets than can be produced according to the invention, an example of a tablet manufactured in a multilayer tablet press is:

A first granulation comprising drug A enters into a die at a first filling station; a second granulation comprising drug B enters on top of said first granulation at a second filling station; a granulation comprising drug A substantially identical in composition and quantity (weight) to said first granulation enters at a third filling station. After final compression, said tablet is ejected from the die. Each granulation, upon full entry into the die and thereafter, forms a layer, or segment, of the final tablet product. This is referred to as an A-B-A configured tablet.

Ideally, in any of the manufacturing processes employed to form a tablet of the subject invention, there is no mixing of drug or excipients from one segment to another. However, in reality, minimal, inadvertent mixing between different granulations in the formation of layers can occur. Therefore, some mixing is to be expected and does not alter the improvement in the art of creating accurate dosing horn breakable tablets from the invention. If such inadvertent intermixing is disadvantageous for a particular product, e.g., incompatibility of active drugs or granulations in contiguously dispensed segments, an inactive or compatible composition can be interposed between the incompatible segments in order to reduce or prevent this intermixing. Different granulations may be of the same or different colors. Wet granulations are often preferred to limit transfer of material from one granulation to another. Direct compression of powder is also a preferred manufacturing technique.

Tablets of the invention are preferably uncoated, but can be coated with conventional coatings for aesthetic or functional or other purpose. However, these coatings are not regarded as a “layer” or “segment” of the tablets of the subject invention. These coatings do not significantly alter the release kinetics of the drug or drugs of the tablets of the invention.

The tablets of the invention are preferably broken transversely in order to realize their benefits or advantages. They may be broken in standard ways, according to the invention such as either by applying force manually (or “by hand” as the term is commonly understood) to cause the tablet to break at a desired location, or by use of an instrument, such as a cutting edge, to apply force directly to a separation mark provided in a desired breaking region.

Separation marks are intended to guide optional tablet breaking in the usual manner that is well known with scores, so that, if tablet breaking is desired, force can be applied to break the tablet at or about the separation mark in a direction that is substantially perpendicular to the surface on which it is desired that breakage of the tablet will be initiated. The tablet according to the invention may be broken either by applying force manually or by an instrument such as a cutting edge directly to the separation mark, or to other areas of the tablet, such as the outer segments, to cause the tablet to break at or about the separation mark and in the direction of the separation mark.

The separation mark or marks may comprise one or more of the following:
(a) a score in a side wherein said score is not oriented vertically;
(h) indicia on at least one side or lateral face of the tablet that indicates or locates a desired breaking region of said tablet;
(c) a band which is located on one segment or at an interface of two segments; or
(d) an inner segment of said tablet in which a first lower and a second upper segment have the same color and contain either the same drug in a pharmacologically effective quantity or both lack a pharmacologically effective quantity of any drug, and the third, inner or interposed segment that has a different color from said first segment and has either the same drug as said first segment when said first segment has a pharmacologically effective quantity of a drug or has no pharmacologically effective quantity of a drug when said first segment lacks a pharmacologically effective quantity of any drug.

En another preferred embodiment, the subject invention concerns a controlled release compressed pharmaceutical tablet that has two or more segments, wherein a first segment includes a pharmacologically effective amount of a drug or drugs and has a deep score that extends up to about 50% or greater into said first segment. More preferably, in one embodiment, the score can be formed from 70% to 99.5% of the distance from a surface of said first segment towards an opposite face (surface) of said first segment having on said opposite face, an adjoining second segment. In an alternative embodiment, the score is formed completely through the first segment and can extend into the second, adjoining segment. In a preferred embodiment, said second segment has an undetectable amount of drug up to a maximum of 80% of the concentration of the drug in said first segment.

Another preferred embodiment of the invention utilizes a variation on the above, for example: A first granulation comprising hydrochlorothiazide (HCTZ) enters the die, followed by an inactive granulation entering the die twice, followed at the fourth and final filling station by a controlled release granulation comprising metoprolol (a beta-blocker). After final compression, a tablet consisting of three segments (formed from four layers) has been created. The simple segment formed from the first granulation is the bottom layer, the layers formed from inactive excipients are the two inner layers and together, after tablet formation, make up the middle (inner) compound segment, and the final granulation comprises the top layer, which after final compression is denoted the top segment, which is a simple segment as defined herein. Thus all dimensions and directions herein relate to the method of manufacture of the tablet. This preferably taller-than-wide tablet may contain some amount of HCTZ in the middle and top segments, and may contain some amount of metoprolol in the middle and bottom segments.

After breaking the above extended release metoprolol/hydrochlorothiazide (HCTZ) tablet entirely through the middle segment, two tablettes are formed. One tablette contains primarily the full, therapeutically effective quantity of HCTZ and may contain some amount, preferably a trace amount, of metoprolol; the other contains primarily the full amount of metoprolol and may contain some amount, preferably a trace amount, of HCTZ, plus some quantity of said middle segment. Important therapeutic benefits in terms of dosage adjustment, side effect management, and the like are obtained from the above tablet design and optional ability to substantially completely create two individual dosage forms from the combination product.

The effective height in the case of beveling or cupping of segments, as easily reflected in the shape of the top of the tablet, is always less than the height of the separating or interposed segment through which breaking is intended to occur. The height of an interposed segment is the vertical distance from its highest point to the highest point of the contiguous superiorly disposed segment.

Another embodiment of the subject invention comprises a bilayer tablet, and preferably comprising unitary segments. Production may involve first allowing a granulation containing active drug into a die that has an embossed lower punch, so that said granulation forms an undivided layer indented from below by said embossing. Said embossing is not limited in its pattern. After optional and preferred tamping, an inactive granulation enters the die and after optional pre-compression, a tablet is formed by final, full-force compression. This compression pushes the first, lower layer almost to the level of the uppermost aspect of the embossing, so that an especially deep score may be produced. Each granulation, after entry into the die, forms a layer. After final compression of the tablet, each layer may also be referred to as a segment of the tablet. Except for inadvertent mixing between granulations, the upper segment is inactive, so that tablet breaking may occur substantially through the inactive segment, thus providing substantial improvement over existing methods of scoring tablets from the standpoint of accuracy of subdividing a dose. Less preferably, the second granulation could contain a diluted quantity of the active ingredient or ingredients comprising said first granulation. Such a maneuver would be useful if it were difficult to place adequate drug substance entirely within said first granulation.

Another preferred embodiment is as follows. A first active granulation enters the die onto an embossed lower punch and is tamped. A second, inactive granulation enters the die at the second filling station and again at the third filling station, and is optionally and preferably tamped after each of said granulations enters said die. At a fourth filling station, a different granulation from the first enters the die, is optionally and preferably tamped, and then final compression takes place, pushing said first granulation lower into the die so that the uppermost part of said first granulation remains above the uppermost part of said embossing. Thus, said first granulation has formed an undivided layer.

In this example, the use of two identical granulations to form two layers that are compositionally substantially identical may be useful to form one tall segment. Such a segment, whether formed from two or more substantially identical inactive granulations or ones comprising an active drug or drugs, is called a compound segment herein. The utility of the dosage form is that it allows different active drugs to primarily be placed in opposite ends of a “taller than wide” tablet, so that both drugs may be given together in a whole tablet, but said tablet also may be broken through a middle segment to create two tablettes comprising substantially different drugs (ignoring any inadvertent mixing between granulations). The current invention is most usefully employed after such optional tablet breaking through said middle segment, after which the first segment may then be itself subdivided if desired to create a plurality of accurately dosed tablettes.

The above example could as easily utilize a granulation compositionally substantially identical to said first granulation to enter (again) at the fourth filling station. Further segments could be added as a fifth segment and beyond, technical capacity for tablet production being the limiting factor. Furthermore, said second segment could comprise an active drug, or a mixture of the drug or drugs present in both the first and third segments in the example above, and the utility of the invention would persist, though relevance in medical or veterinary practice would relate to the nature of the drug or drugs in said middle segment.

Another preferred embodiment is as follows. A first granulation comprising a drug enters into a tablet die. An embossing that is 0.3 mm high bisects the lower punch. A second, inactive granulation enters said die above said first granulation. The tablet is compressed. The first segment is one (1.0) mm high after final compression. Thus the score is 30% of the way through said first segment. The tablet has immediate release characteristics. The tablet is novel but lacks substantial advantages over tablets known in the art that lack a substantially inactive segment, but the second segment does provide structural support for the tablet, so there may be some advantage.

The invention thus teaches novel methods of manufacture of deep scores within pharmacologically active parts of the tablet. Preferred methods of manufacture of the tablets of the invention that utilize an embossed bottom punch to produce the scored segment that is the subject of the invention utilize an upper punch that does not have any embossing, or else has an embossing of a small vertical dimension, above the embossing present on and extending upwards from the base of said lower punch.

A different mode of manufacture may be employed, using an embossed upper punch and a preferably flat-faced lower punch. In this technique, a most preferred tablet of the invention may be produced as follows. A first, inactive granulation enters the die and is optionally tamped. A second granulation comprising drug then enters the die, is optionally tamped, and final compression occurs. Some amount of drug lies under the lower part of said embossing but the bulk of second granulation is apart from the breaking area, and thus when and if force is applied in a conventional, vertical fashion to the lowest aspect of the score, highly accurate tablet breaking will take place with respect to the active drug.

Tablets of the above design are not limited to two segments. A segment represents a contiguous part of a tablet of the invention that is formed from one granulation entering the tablet die at a time, with exceptions such as the following: If two successive granulations comprised the same active drug and similar excipients, then when compressed, they would comprise one segment. If, however, two different active drugs, such as different active drugs or different salts of the same active drug, were compressed onto each other, they would comprise two segments. Granulations comprising the same active drug but with dissimilar excipients would comprise two segments if one granulation were compressed onto another.

Benefits of the invention are not limited to tablets of any specific number of active ingredients. All segments containing an active ingredient may contain the same drug, or segments may contain different drugs.

In order to fully realize the benefits of the invention, a score may be placed into a segment (or interface between segments) of the tablet. This score may be formed in an inner segment with a file in a substantially horizontal manner, so that breaking the tablet through said score could lead to breaking through the inner segment while leaving the outer segments intact.

A further embodiment includes a unitary segment configuration wherein the embossed or post-production score is configured completely through an outer, e.g., bottom segment. In addition, similar means of marking tablets may be followed such as by causing an edible ink to be placed on the tablets, thus delineating a desired region of the tablet, such as its middle segment. Such application is well known in the art. Other means of applying indicia are contemplated as within the scope of the invention.

Preferred tablets of the invention often use a height and an effective height that are both over 4 mm, and may exceed 6 mm. Lesser heights and effective heights are utilized when needed due to size constraints on the tablet.

Examples of specific embodiments of the invention are described with reference to the drawings accompanying this disclosure. The drawings depict vertical cross-sectional views of tablets and tablettes of the invention. Tablets are depicted as if they were in the die, so that the top of the tablet as it is oriented on the page corresponds with the top of the tablet in the die. In other words, the top segment of the tablet as viewed contains the last granulation to enter the die. Tablettes are depicted as they would have been in the die before they were separated from the intact tablet. Shaded areas represent segments derived from active granulations, i.e., those which contain a drug; clear (plain) areas represent segments derived from inactive granulations, i.e., those formulated with no active drug.

“Front views” refer to a cross-sectional view of a tablet that has a theoretical geometric plane passed through the tablet relative to a side which is arbitrarily designated as the front. Figures labeled as “side view”, which also have a corresponding “front view”, are taken as a cross-section through the whole tablet from the right side of a front view i.e. a side view is a cross-section that is taken by passing a plane through the vertical axis of the whole tablet at a 90° angle to the cross-sectional front view. Each front view represents a schematic cross-section that passes through the midpoint of the horizontal cross-section as measured from the front of the tablet to the back of the tablet or tablette. The front view is also parallel to the major axis of the tablet (e.g., for a tablet with a rectangular (but not square) transverse cross-section, the longer side of the perimeter is parallel with the plane that depicts the cross-sectional, front view). That plane is located half-way between the front and back surfaces of said tablet. Drawings are of tablets that have a rectangular but not square horizontal cross-section at the vertical mid-point of the tablet.

Segments containing pharmacologically active amounts of a drug or drugs are shown crosshatched; pharmacologically ineffective segments are shown plain (clear, without crosshatching or stippling). For consistency, tablettes are depicted in the same orientation as the tablets from which they are formed, although tablettes are created after tablet ejection from the die. Dotted lines in the tablets depicted in the figures may represent printed marks or other indicia, or scores that are present on or in the surface of the tablet and, if they represent a score, said score does not extend deeply enough into the tablet to appear in the cross-sectional front view. The transverse dotted lines reflecting scores shown in the Figures imply no intention to limit the depth of any scores of the tablets of the invention. Horizontal dotted lines on the front views that represent the surface scores are schematic, and do not necessarily represent the full vertical extent of a score, printed mark, or the like.

Separation marks in the tablets depicted in the Figures are depicted as scores that are present on or in the surface of the tablet and that do not extend deeply enough into the tablet to appear in the cross-sectional front views are depicted in the drawings as dotted lines to reflect the location of said scores on or in the surface of the tablet (not shown). It is to be understood that the depth of a separation mark or other score may be deeper than one-half the widest cross-section of the tablet in a particular embodiment, and thus the transverse dotted lines reflecting scores that are separation marks shown in the Figures imply no intention to limit the depth of any scores of the tablets of the invention. Similarly, the tablets shown that contain scores do not limit the width or extent of said scores. The horizontal dotted lines on the front views that represent the surface scores are schematic, and do not necessarily represent the full vertical extent of the score. (Perforations or discontinuous scores through the width or depth of the tablets are not depicted herein, but remain within the scope of the invention, as are other marks on or physical changes to the tablet that create a separation mark.) Any scores or printed indicia that serve as separation marks are for convenience herein assumed to be on the front surface of the tablet, which is arbitrarily chosen from a vertically-oriented surface of the tablets. The “side view” of a tablet is a cross-sectional view of the tablet rotated 90 degrees from the front view. No dimension of the separation marks is limited by their depiction as dotted lines in any figure.

FIG. 1 shows a three-segment tablet of the subject invention. Preferably, the top segment A comprises a controlled release composition that contains a drug or drugs. The controlled release composition may preferably be a matrix composition. The middle segment I preferably comprises a composition that is intended to be broken through when a partial dose is desired, and prevents egress of drug therethrough when the tablet or a portion thereof is ingested into the body. The bottom segment B can comprise an identical composition as in segment A or I or can comprise a different immediate release or controlled release composition. In a most preferred embodiment, the dosage form shown in FIG. 1 comprises a top segment A which is a matrix composition comprising a drug, a middle segment which can be broken through to provide a partial dose from said dosage form and is a formulation that prevents egress of drug therethrough, and a bottom segment B which is a matrix composition comprising a drug and is substantially identical to said composition of the top segment.

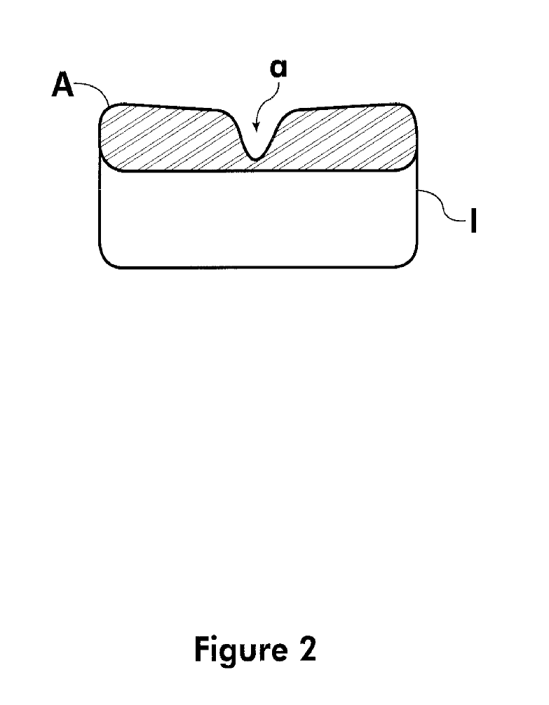

FIG. 2 shows a cross-section of a two-segment tablet of the subject invention comprising a deep score a. Preferably, the top segment A comprises a controlled release composition that contains a drug or drugs. The controlled release composition may preferably be a matrix composition. The bottom segment, 1, preferably comprises a composition that is intended to be broken through when a partial dose is desired, and prevents egress of drug therethrough when the tablet or a portion thereof is ingested into the body. In a most preferred embodiment, the dosage form shown in FIG. 2 comprises a top segment A which is a matrix composition comprising a drug, and a bottom segment which can be broken through to provide a partial dose from said dosage form and is a formulation that prevents egress of drug therethrough.

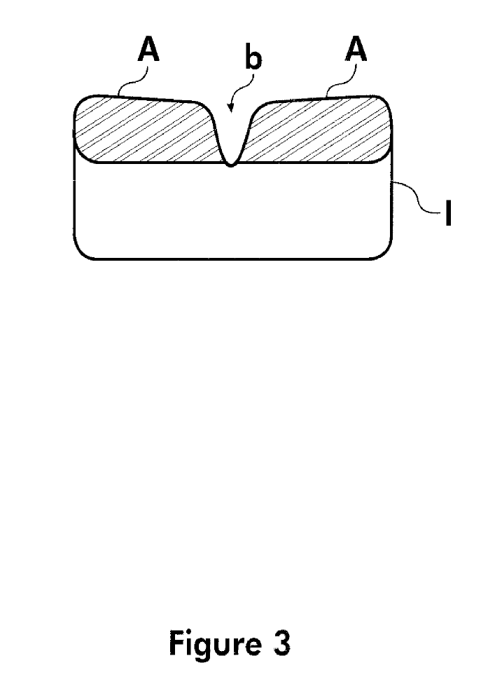

FIG. 3 shows a cross section of a two-segment tablet of the subject invention as in FIG. 2, but having a score b which is formed completely through the active segment and extends into the inactive segment.

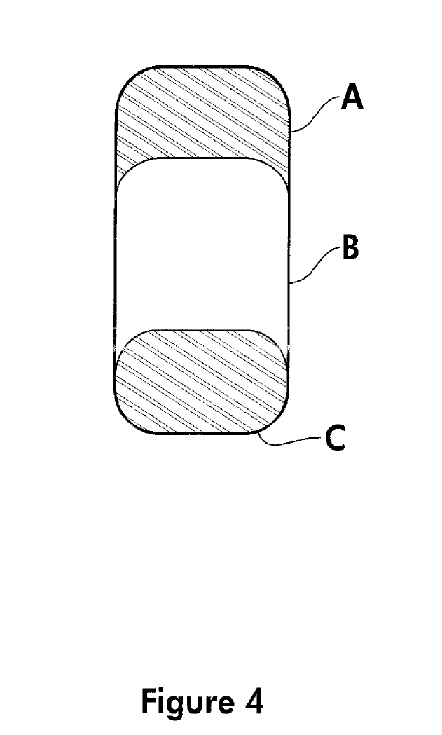

FIG. 4 shows an alternative embodiment of a three-segment tablet of the subject invention. Preferably, the top segment A comprises a controlled release composition that contains a drug or drugs. The controlled release composition may preferably be a matrix composition. The middle segment B preferably comprises a composition that is intended to be broken through when a partial dose is desired. In one embodiment, the composition of segment B can comprise an impermeable or insoluble composition that can prevent egress of drug therethrough when the tablet or a portion thereof is ingested into the body. The middle segment B can comprise active drug or can be an inactive composition. The bottom segment C can comprise an identical composition as in segment A or B or can comprise an immediate release or controlled release composition that contains a different drug or drugs.

In a most preferred embodiment, the dosage form shown in FIG. 4 comprises a top segment A which is a matrix composition comprising a drug for controlled release of the drug from the matrix composition. A preferred drug is niacin. The middle segment B preferably comprises a second drug, such as a non-steroidal anti-inflammatory drug (NSAID), e.g., acetylsalicylic acid (aspirin) in an immediate release formulation. Other NSAIDs that can be used in place of aspirin include but are not limited to piroxicam, celocoxib, ibuprofen, and indomethacin. The middle segment composition can be broken through to provide a partial dose of the drugs contained in the whole dosage form. Alternatively, the middle segment can be a formulation comprising a composition that prevents egress of drug therethrough. The bottom segment C is preferably a drug in a matrix composition that is substantially identical to the composition of the top segment A.

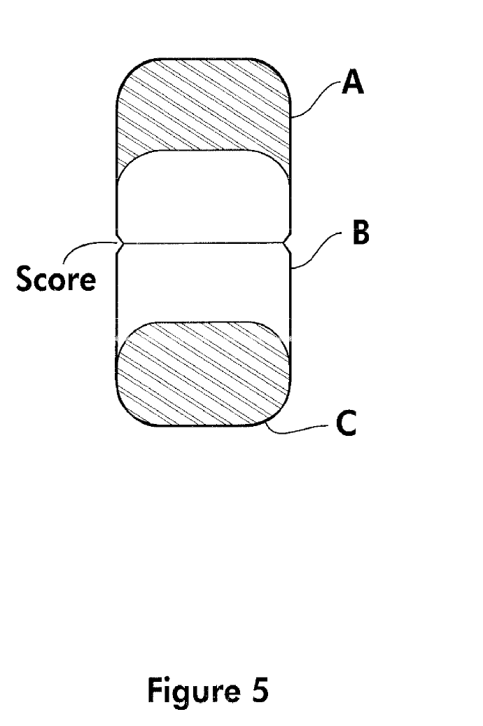

Alternatively, the dosage form of the subject invention can comprise the first and second active drugs in separate top and bottom segments, A and C, and further comprising an inactive segment B interposed therebetween. In one preferred embodiment, the dosage form comprises a score, and can be placed in the middle segment B, as shown in FIG. 5.

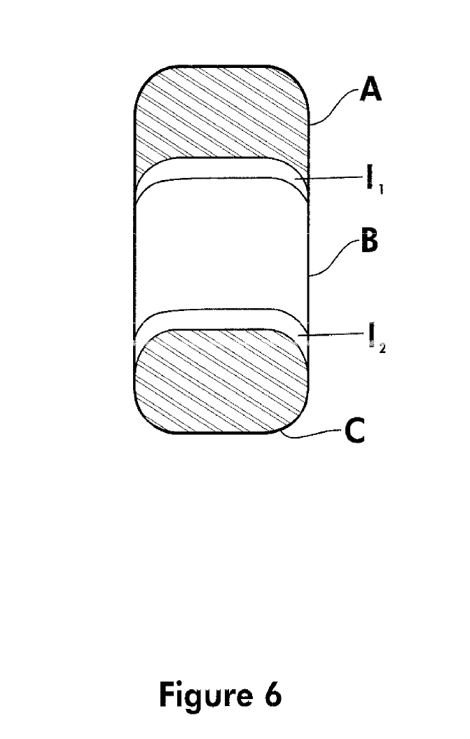

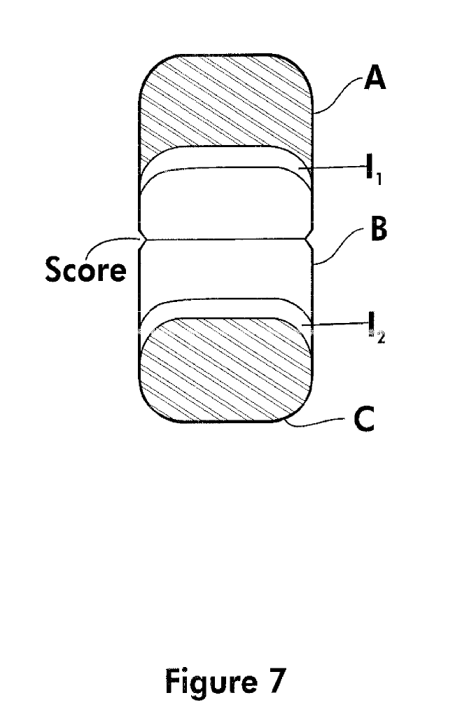

FIG. 6 shows a variation of the three-segment tablet of FIG. 1 wherein the tablet comprises three active segments A, B, and C, as described above for FIG. 4, plus two inactive (substantially drug free) segments I₁ and I₂. One inactive segment is interposed between and separates each of the three active segments. Preferably, the inactive segments comprise compositions that form barrier layers and can prevent or retard egress of drug therethrough from a contiguous active segment. An embodiment of the five-segment tablet of FIG. 6, having a score in the middle active segment is shown in FIG. 7.

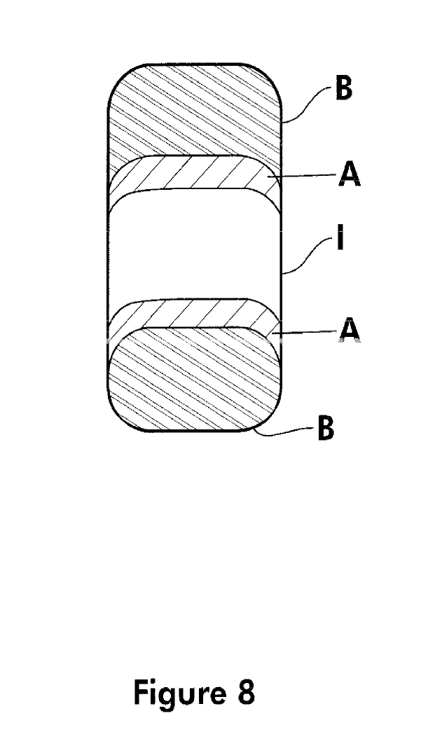

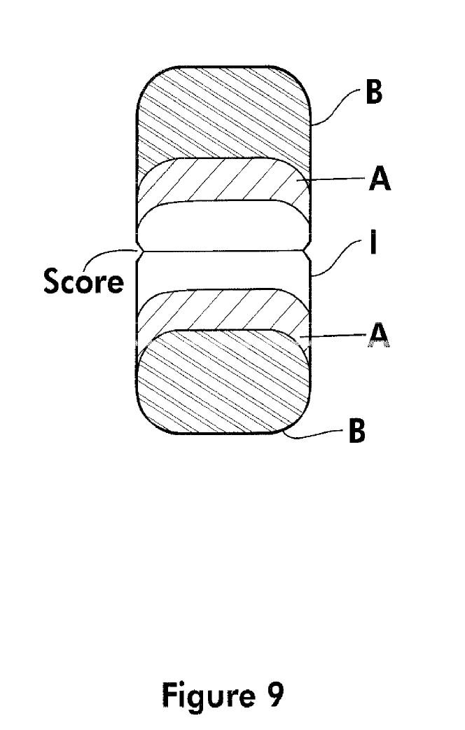

FIG. 8 shows a further embodiment of a live-segmented tablet of the invention comprising two different active compositions, A and B, forming the bottom two segments. Two different active compositions (also shown as A and B, being preferably substantially identical to the respective bottom two segments) forming the top two segments, and an inactive barrier segment interposed between the top two and bottom two segments. In a preferred embodiment, segment A comprises an NSAID, such as aspirin, and segment B comprises a drug such as niacin. The inactive middle segment preferably comprises a composition that can be broken through and forms a barrier layer to prevent or retard egress of drug therethrough from a contiguous active segment B. An embodiment of the five-segment tablet of FIG. 8, having a score in the middle active segment is shown in FIG. 9.

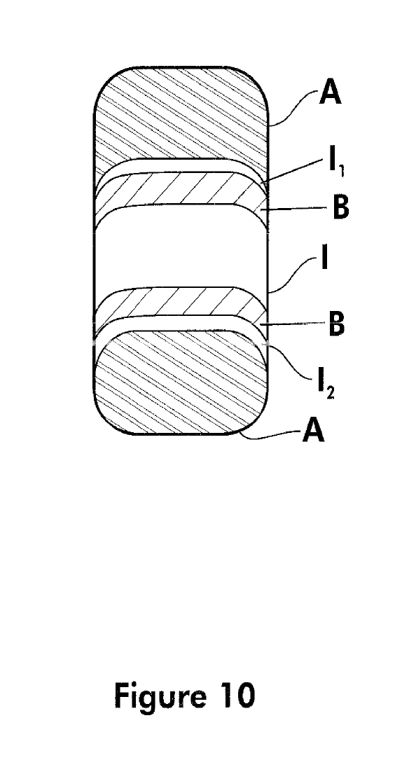

FIG. 10 shows a seven-segment variation of the five-segment tablet of FIG. 8 where the two top active segments A and B are separated from one another by an additional interposed inactive segment, I₁ and the two bottom active segments are separated by an additional interposed inactive segment, I₂. The inactive segments I₁ and I₂ can comprise any pharmaceutically acceptable ingredients, and preferably comprise a substantially drug-free immediate release composition.

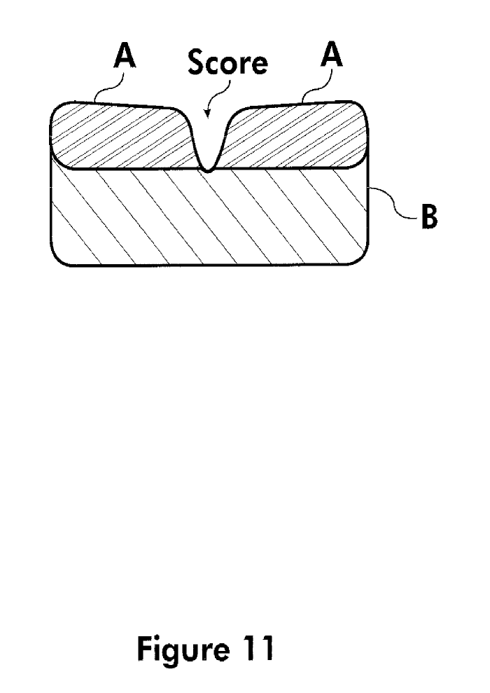

FIG. 11 shows a cross section of a bi-layer tablet of the subject invention comprising active segments A and B. A deep or complete score is preferably formed completely through, or substantially completely through, active segment A and into a second active segment B. Active segment B can serve as a base layer or segment for the tablet. In a preferred embodiment, segment A comprises a drug such as niacin, and segment B comprises a drug such as an NSAID, e.g., aspirin.

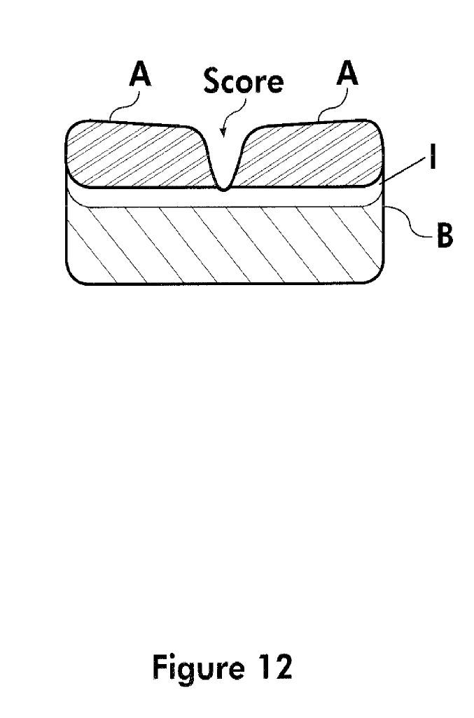

FIG. 12 shows a variation of the bi-layer tablet of FIG. 11 wherein a third inactive or harrier segment f is interposed between the first and second active segments A and B. The inactive middle segment preferably comprises a composition that forms a barrier layer to prevent or retard egress of drug therethrough from a contiguous active segment A.

### DESCRIPTION OF MANUFACTURE OF PREFERRED EMBODIMENTS

Hydrophilic matrix systems are among the most widely used means for controlled drug delivery in a solid oral dosage form. Formulation and production of matrix systems are conventional in the art of pharmaceutical tablet manufacture. Tablets can be manufactured with commercially available equipment and conventional processing methods.

Below are active formulas that can be used to construct matrix controlled-release tablets containing active formulations, and may contain inactive composition formulations in accordance with the subject invention.

### Example 1

### a. Metoprolol Succinate Active Composition (Active #1)

| Ingredient | Weight (mg) |
|---|---|
| Methocel K4MP | 25.000 |
| Metoprolol tartrate | 6.250 |
| Lactose | 93.125 |
| Magnesium Stearate | 0.625 |

### b. Inactive Composition

| Ingredients for middle segment: | Mg. |
|---|---|
| Dibasic calcium phosphate anhydrous | 158.59 |
| Magnesium stearate | 2.79 |
| PVP K-30 | 2.62 |
| | 164.00 |

The compositions can be prepared using, for example, the following mixing procedures:
i. Powder Mixing (Twin-Shell Blender):
Drug, excipient/filler and polymer are charged into the twin-shell blender and mixed for 10 minutes. Magnesium stearate is added and mixed for two minutes; or
ii. Powder Mixing (High-Shear Mixer):
Drug and polymer are charged into the high shear mixer and mixed for 1 minute at 200 rpm main blade speed and 1000 rpm chopper speed to help ensure homogeneity. After this premix step, magnesium stearate is added and mixed at the same rates for two minutes.

### c. Tablet Preparation

Mixes are tableted using a 27-station Stokes tri-layer rotary tablet press equipped with 0.131 inch by 0.3222 inch oval, concave tablet punches. The bottom segment is introduced first into the die. The tablet weight is adjusted between 300 mg to 450 mg depending on the Formula chosen. Tablets so made are about 10 mm tall; the inactive middle segment varies from 5-8 mm in height and a width of 4 mm. The applied compression force is about 6000 lb (26.6 kN).

### d. Tabletting Instructions

1. Place the Matrix powder mix for Active Drug (Active #1) in hopper 41.
2. Place the Inactive composition powder for second segment (layer #2) in hopper #2.
3. Place the Matrix powder mix for Active Drug (Active #1) for Layer 43 in hopper #3.
4. Compress the segments to desired weight (tablets for Active #1 should form a soft compact).
5. Compress Active #1 & Inactive layer #2 tablets to desired combined weight of Active #1 and layer #2 weight (tablets should form a soft compact).
6. Compress the tri-layer tablet to the desired total tablet weight (Active #1 weight+layer #2 weight+Active # 1 (layer #3) weight). Tablet should be compressed at desired hardness.

### Example 2

A formulation of an alprazolam active composition can be prepared according to the following formula:

### Alprazolam Active Composition

| Ingredient | Weight (mg) |
|---|---|
| Methocel K4MP | 20.00 |
| Alprazolam | 1.25 |
| Filler | 18.25 |
| Microcrystalline Cellulose | 10.00 |
| Silicon Dioxide | 0.25 |
| Magnesium Stearate | 0.25 |

The alprazolam active formulation can be prepared as described in Example 1, with appropriate modifications made for weights and amounts of ingredients as would be understood by a person of ordinary skill in the art. Tabletting instructions in accordance with those provided in Example 1 can be used for preparation of an alprazolam product.

### Example 3

A formulation of a promethazine active composition can be prepared according to the following formula:

| Ingredient | Weight (mg) |
|---|---|
| Methocel K4MP | 25.000 |
| Promethazine | 6.250 |
| Lactose | 93.125 |
| Magnesium Stearate | 0.625 |

The promethazine active formulation can be prepared as described in Example 1, with appropriate modifications made as would be understood by a person of ordinary skill in the art. Tabletting instructions in accordance with those provided in Example 1 can be used for preparation of a promethazine product.

### Example 4

A tablet can be made which has three segments: (1) an active top or upper segment comprising niacin in a matrix formulation, and (2) an active lower or bottom segment comprising niacin in a matrix formulation, the top and bottom segments being separated by (3) a middle segment comprising aspirin in a conventional immediate release formulation. A Stokes 27-station tri-layer rotary tablet press can be used for layering the segments of the tablet.

All formulations comprise directly compressible compositions, and are manufactured using conventional techniques and processes, as are well known in the pharmaceutical manufacturing art. For example, powder blend formulations can be performed in a Patterson-Kelly “V” blender. Coatings can be applied by any means commonly known in the industry, however, if the anti-sticking agent is to be dusted onto the cores during the coating process, it is preferred to use a rotary granulator or pan coater for the coating process. If the anti-sticking agent is applied by suspending it in the coating solution, it is preferred to use a fluidized bed coater or rotary granulator for the coating process.

The tablets are compressed using, for example, 0.131 inch by 0.3222 inch oval, concave tablet punches to a hardness of 35 kiloponds. The bottom segment is introduced first into the die. The tablet weight is 300 mg. Tablets so made are about 11 mm tall; the inactive middle segment varies from about 5-8 mm in height and a width of about 4-6 mm.

Examples of a niacin/xanthan gum tablet formulations and their method of preparation are as follows:

Niacin Base Granulation: Niacin (Nicotinic Acid) Roche 97.0% Maltodextrin M-100 3.0% The niacin was charged into a fluid bed agglomerator and the maltodextrin was sprayed over as a 15% aqueous solution to effect agglomeration and compressibility with concomitant good flow characteristics. The final granulation was sized −20 mesh, U.S. sieve size.

Niacin Base Granulation 61.9%, Xanthan Gum (Keltrol SF) 37.4% Stearic Acid 0.7%. The components were well mixed and compressed on caplet punches at a weight of 840 mg/tablet at a hardness of 12 kp.

Each 840 mg Tablet yields:
Niacin 504.4 mg
Xanthan Gum 314.2 mg
Stearic Acid 5.9 mg
Maltodextrin 15.5 mg

The compositions incorporating xanthan gum exhibit satisfactory sustained release of the active ingredients therein into the gastro-intestinal tract.
Tabletting Instructions
   1. Place the powder for niacin active layer in hopper #1.
   2. Place the powder for aspirin active layer in hopper #2.
   3. Place the powder for niacin active layer in hopper #3.
   4. Compress layer #1 tablets to desired weight (tablets for layer #1 should form a soft compact).
   5. Compress layer #1 & layer #2 tablets to desired combined weight of layer #1 and layer #2 weight (tablets should form a soft compact).
   6. Compress the tri-layer tablet to the desired total tablet weight (layer #1 weight+layer #2 weight+layer #3 weight) Tablet should be at desired hardness.

### Example 5

Formulations comprising therapeutic amounts of phenytoin can be prepared using the techniques and procedures of any of Examples 1-5, above, with appropriate modification as would be apparent to a person of ordinary skill in the art of pharmaceutical formulation and tablet production.

### Example 6

Formulations comprising therapeutic amounts of venlafaxine can be prepared using the techniques and procedures of any of Examples 1-5, above, with appropriate modification as would be apparent to a person of ordinary skill in the art of pharmaceutical formulation and tablet production.

It will thus be seen that the objects set forth above, among those made apparent from the preceding description, are efficiently attained and, since certain changes may be made in the above constructions without departing from the spirit and scope of the invention, it is intended that all matter contained in the above description shall be interpreted as illustrative and not in a limiting sense.

While this invention has been illustrated and described in what are considered to be the most practical and preferred embodiments it will be recognized that many variations are possible and come within the scope thereof, the appended claims therefore being entitled to a full range of equivalents.

1. A dosage form comprising a plurality of segments, said dosage form comprising a first segment comprising a drug in a controlled release composition and a second segment comprising a composition which prevents or retards release of said drug from said first segment, wherein said second segment is breakable such that the first segment provides an accurately divided dose following breakage of said second segment.

2. The dosage form of claim 1 wherein said dosage form is a tablet.

3. The dosage form of claim 1 wherein said composition of said second segment prevents or retards the release of said drug from said first segment both in the unbroken tablet and when the tablet is broken through said second segment.

4. The dosage form of claim 1 wherein said controlled release composition is a selected from the group consisting of a compressed bead or pellet composition and a matrix composition.

5. The dosage form as in claim 1 wherein said dosage form comprises a separation mark selected from a printed mark, a gelatin band, a color designation, and a score.

6. The tablet of claim 2, said tablet consisting essentially of two segments.

7. The tablet of claim 2, said tablet consisting essentially of three segments.

8. The tablet of claim 2, said tablet consisting essentially of five segments.

9. The dosage form as in claim 5 wherein said score extends at least 70% through the first segment.

10. The dosage form of claim 5 wherein said score extends more than 95% through said first segment.

11. The dosage form of claim 1 wherein said second segment prevents substantially all of the drug present in the first segment from leaving said first segment through said second segment after ingestion by a user.

12. The dosage form of claim 1 in which said second segment prevents at least 75% of the drug present in the first segment to leave the first segment through said second segment after ingestion by a user.

13. The dosage form of claim 1 wherein a pharmacokinetic or drug release profile of a drug present within said first segment in a whole tablet is substantially identical to a pharmacokinetic or drug release profile of said drug after breaking of said whole tablet through said second segment.

14. The tablet of claim 7 comprising controlled release compositions in each segment that comprises a pharmaceutically effective amount of a drug.

15. The tablet of claim 14 wherein the tablet comprises two controlled release segments that are compositionally substantially identical.

16. The tablet of claim 14 in which said first segment is above and said third segment is below said second segment as positioned in a tablet die during manufacture.

17. The tablet of claim 14 wherein said second segment is contiguous with at least one of said first and third segments.

18. The dosage for of claim 1 wherein at least one segment of said dosage form is coated.

19. The dosage form of claim 18 wherein said coating does not substantially affect the release kinetics of a drug present within said first segment.

20. The dosage form of claim 5 wherein said score is created after said dosage form is formed.

21. The dosage form as in claim 1 said dosage form further comprising a third segment interposed between said second segment and said first segment, and said interposed segment is substantially insoluble in aqueous solution.

22. The dosage form of claim 1 wherein said drug is selected from the group consisting of alprazolam, aspirin, piroxicam, celocoxib, ibuprofen, indomethacin didanosine, lithium carbonate, metoprolol, nicotinic acid or niacin, phenytoin, potassium chloride, theophylline, and venlafaxine, or a salt, hydrate, polymorph, derivative, or prodrug thereof.

23. A method of using a dosage form of claim 1 wherein said dosage form is provided as a whole tablet, said method comprising:
a) Breaking said whole tablet through the second segment to provide a tablet portion comprising the first segment containing substantially the same dose as in said first segment prior to breaking of the second segment, and
b) Administering to a patient the tablet portion comprising the first segment.

24. The method of claim 23 wherein said dose in said first segment after breaking is more than 90% of the dose prior to breaking.

25. The method of claim 23 wherein said dose in said first segment after breaking is more than 95% of the dose prior to breaking.

26. The method of claim 23 wherein said dose in said first segment after breaking is more than 99% of the close prior to breaking.

27. The method of claim 23 wherein said dose in said first segment after breaking is more than 99.5% of the dose prior to breaking.

28. The method of claim 23 wherein said dose in said first segment after breaking is more than 99.9% of the close prior to breaking.

29. The dosage form of claim 1 wherein said second segment provides a harrier to egress of drug from the first segment at an interface where the second segment is adjoined directly or indirectly to said first segment, wherein said barrier to egress of drug prevents 50% or more of said drug from egressing from said first segment at said interface.

30. The dosage form of claim 29 wherein said egress of drug is prevented more than 75%.

31. The dosage form of claim 29 wherein said egress of drug is prevented more than 90%.

32. The dosage form of claim 29 wherein said egress of drug is prevented more than 95%.

33. The dosage form of claim 1 further comprising a fourth segment interposed between said first and second segments, and a fifth segment interposed between said second and third segments.

34. The dosage form of claim 33 wherein said fourth and fifth segments comprise inactive or substantially drug-free compositions.

35. The dosage form of claim 34 wherein said fourth and fifth segments comprise a barrier layer which prevents or retards drug from leaving or egressing from an active segment contiguous therewith.

36. The dosage form of claim 1 wherein said first and third segments comprise the same active composition, said second segment comprises an inactive composition, said dosage form further comprising a fourth segment contiguous with said first segment and a fifth segment contiguous with said third segment.

37. The dosage form of claim 36 wherein said inactive composition of said second segment is a barrier composition which prevents or retards drug from leaving or egressing from an active segment contiguous therewith.

38. The dosage form of claim 36 further comprising a sixth segment interposed between said first and fourth segment and a seventh segment interposed between said second and fifth segment.

39. The dosage form of claim 38 wherein said sixth and seventh segments comprise an inactive composition.
